(19) United States
(12) Patent Application Publication
(10) Pub. No.: US 2006/0079794 A1
(43) Pub. Date: Apr. 13, 2006

(54) Monitoring device, method and system

(75) Inventors: Steve Liu, San Diego, CA (US); Donald Brady, Las Vegas, NV (US); Matthew J. Banet, Del Mar, CA (US); Sammy I. Elhag, San Diego, CA (US)

Correspondence Address: Michael A. Catania;Impact Sports Technologies, Inc., 2101 Plaza Del Dios, Las Vegas, NV, 89102, US

(73) Assignee: Impact Sports Technologies, Inc.

(21) Appl. No.: 11/046,274

(22) Filed: Jan. 29, 2005

Related U.S. Application Data
(60) Provisional application No. 60/613,785, filed on Sep. 28, 2004.

(51) Int. Cl. A61B 5/02

(52) U.S. Cl. 600/502

(57) ABSTRACT

A monitoring device (20) and method (200) for monitoring the health of a user is disclosed herein. The monitoring device (20) is preferably a glove with an embedded optical sensor (30), a circuitry assembly (35) a display member (40) and a control component (43). The monitoring device (20) preferably displays the following information about the user: pulse rate; blood oxygenation levels; calories expended by the user of a pre-set time period; target zones of activity; time; distance traveled; and dynamic blood pressure.

### CROSS REFERENCES TO RELATED APPLICATION

The Present Application is a continuation-in-part application of U.S. Provisional Application No. 60/613,785 filed on Sep. 28, 2004.

### STATEMENT REGARDING FEDERALLY SPONSORED RESEARCH OR DEVELOPMENT

Not Applicable

### BACKGROUND OF THE INVENTION

1. Field of the Invention

The present invention is related to health monitoring devices. More specifically, the present invention relates to a glove for monitoring a user's vital signs.

2. Description of the Related Art

There is a need to know how one is doing from a health perspective. In some individuals, there is a daily, even hourly, need to know one's health. The prior art has provided some devices to meet this need.

One such device is a pulse oximetry device. Pulse oximetry is used to determine the oxygen saturation of arterial blood. Pulse oximeter devices typically contain two light emitting diodes: one in the red band of light (660 nanometers) and one in the infrared band of light (940 nanometers). Oxyhemoglobin absorbs infrared light while deoxyhemoglobin absorbs visible red light. Pulse oximeter devices also contain sensors that detect the ratio of red/infrared absorption several hundred times per second. A preferred algorithm for calculating the absorption is derived from the Beer-Lambert Law, which determines the transmitted light from the incident light multiplied by the exponential of the negative of the product of the distance through the medium, the concentration of the solute and the extinction coefficient of the solute.

The major advantages of pulse oximetry devices include the fact that the devices are non-invasive, easy to use, allows for continuous monitoring, permits early detection of desaturation and is relatively inexpensive. The disadvantages of pulse oximetry devices are that it is prone to artifact, it is inaccurate at saturation levels below 70%, and there is a minimal risk of burns in poor perfusion states. Several factors can cause inaccurate readings using pulse oximetry including ambient light, deep skin pigment, excessive motion, fingernail polish, low flow caused by cardiac bypass, hypotension, vasoconstriction, and the like.

Chin et al., U.S. Pat. No. 6,018,673 discloses a pulse oximetry device that is positioned entirely on a user's nail to reduce out of phase motion signals for red and infrared wavelengths for use in a least squares or ratio-of-ratios technique to determine a patient's arterial oxygen saturation.

Smith, U.S. Pat. No. 4,800,495 discloses an apparatus for processing signals containing information concerning the pulse rate and the arterial oxygen saturation of a patient. Smith also discloses maintaining the position of the LEDs and detectors to prevent motion-artifacts from being produced in the signal.

Another method for using a pulse oximeter to measure blood pressure is disclosed in U.S. Pat. No. 6,616,613 to Goodman for a ‘Physiological Signal Monitoring System’. The '613 Patent discloses processing a pulse oximetry signal in combination with information from a calibrating device to determine a patient's blood pressure.

Chen et al, U.S. Pat. No. 6,599,251 discloses a system and method for monitoring blood pressure by detecting pulse signals at two different locations on a subjects body, preferably on the subject's finger and earlobe. The pulse signals are preferably detected using pulse oximetry devices.

Schulze et al., U.S. Pat. No. 6,556,852, discloses the use of an earpiece having a pulse oximetry device and thermopile to monitor and measure physiological variables of a user.

Malinouskas, U.S. Pat. No. 4,807,630, discloses a method for exposing a patient's extremity, such as a finger, to light of two wavelengths and detecting the absorbance of the extremity at each of the wavelengths.

Jobsis et al., U.S. Pat. No. 4,380,240 discloses an optical probe with a light source and a light detector incorporated into channels within a deformable mounting structure which is adhered to a strap. The light source and the light detector are secured to the patient's body by adhesive tapes and pressure induced by closing the strap around a portion of the body.

Tan et al., U.S. Pat. No. 4,825,879 discloses an optical probe with a T-shaped wrap having a vertical stem and a horizontal cross bar, which is utilized to secure a light source and an optical sensor in optical contact with a finger. A metallic material is utilized to reflect heat back to the patient's body and to provide opacity to interfering ambient light. The sensor is secured to the patient's body using an adhesive or hook and loop material.

Modgil et al., U.S. Pat. No. 6,681,454 discloses a strap that is composed of an elastic material that wraps around the outside of an oximeter probe and is secured to the oximeter probe by attachment mechanisms such as Velcro, which allows for adjustment after initial application without producing excessive stress on the spring hinge of the oximeter probe.

Diab et al., U.S. Pat. No. 6,813,511 discloses a disposable optical probe suited to reduce noise in measurements, which is adhesively secured to a patient's finger, toe, forehead, earlobe or lip.

Diab et al., U.S. Pat. No. 6,678,543 discloses an oximeter sensor system that has a reusable portion and a disposable portion. A method for precalibrating a light sensor of the oximeter sensor system is also disclosed.

Tripp, Jr. et al., U.S. Statutory Invention Registration Number H1039 discloses an intrusion free physiological condition monitor that utilizes pulse oximetry devices.

Hisano et al., U.S. Pat. No. 6,808,473, discloses a headphone-type exercise aid which detects a pulse wave using an optical sensor to provide a user with an optimal exercise intensity.

In monitoring one's health there is a constant need to know how many calories have been expended whether exercising or going about one's daily routine. A calorie is a measure of heat, generated when energy is produced in our bodies. The amount of calories burned during exercise is a measure of the total amount of energy used during a workout. This can be important, since increased energy usage through exercise helps reduce body fat. There are several means to measure this expenditure of energy. To calculate the calories burned during exercise one multiplies the intensity level of the exercise by one's body weight (in kilograms). This provides the amount of calories burned in an hour. A unit of measurement called a MET is used to rate the intensity of an exercise. One MET is equal to the amount of energy expended at rest.

For example, the intensity of walking 3 miles per hour (“mph”) is about 3.3 METS. At this speed, a person who weighs 132 pounds (60 kilograms) will burn about 200 calories per hour (60×3.3=198).

The computer controls in higher-quality exercise equipment can provide a calculation of how many calories are burned by an individual using the equipment. Based on the workload, the computer controls of the equipment calculate exercise intensity and calories burned according to established formulae.

The readings provided by equipment are only accurate if one is able to input one's body weight. If the machine does not allow this, then the “calories per hour” or “calories used” displays are only approximations. The machines have built-in standard weights (usually 174 pounds) that are used when there is no specific user weight.

There are devices that utilize a watch-type monitor to provide the wearer with heart rate as measured by a heartbeat sensor in a chest belt.

The prior art has failed to provide a means for monitoring one's health that is accurate, easy to wear on one's body for extended time periods, allows the user to input information and control the output, and provides sufficient information to the user about the user's health. Thus, there is a need for a monitoring device that can be worn for an extended period and provide health information to a user.

### BRIEF SUMMARY OF THE INVENTION

The present invention provides a solution to the shortcomings of the prior art. The present invention is accurate, comfortable to wear by a user for extended time periods, allows for input and controlled output by the user, is light weight, and provides sufficient real-time information to the user about the user's health.

One aspect of the present invention is a monitoring device for monitoring the health of a user. The monitoring device includes a glove, an optical device for generating a pulse waveform, a circuitry assembly embedded within the glove, a display member attached to an exterior surface of the glove, and a control means attached to the glove.

The glove preferably has a plurality of finger sleeves which have minimal length for the comfort of the user. The glove preferably has a minimal mass, one to five ounces, and is flexible so that the user can wear it the entire day if necessary. The monitoring device allows the user to track calories burnt during a set time period, monitor heart rate, blood oxygenation levels, distance traveled, target zones and optionally dynamic blood pressure.

Another aspect of the present invention is a method for monitoring a user's vital signs. The method includes generating a signal corresponding to the flow of blood through an artery of the user. The signal is generated from an optical device connected to a body of a glove. Next, the heart rate data of the user and an oxygen saturation level data of the user is generated from the signal. Next, the heart rate data of the user and the oxygen saturation level data of the user are processed for analysis of calories expended by the user and for display of the user's heart rate and blood oxygen saturation level. Next, the calories expended by the user, the user's heart rate or the user's blood oxygen saturation level are displayed on a display member attached to an exterior surface of the body of the glove, which is controlled by the user using a control component extending from the body of the glove.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a schematic view of a preferred embodiment of a monitoring device.

FIG. 2 is an isolated view of a display member utilized with the monitoring device.

FIG. 3 is a side view of a monitoring device placed on the hand of a user.

FIG. 4 is palm side view of FIG. 3.

FIG. 5 is a back side view of FIG. 4.

FIG. 6 is an isolated side view of an optical sensor utilized with a monitoring.

FIG. 7 is a top plan view of the optical sensor of FIG. 6.

FIG. 8 is a top plan view of an alternative embodiment of the monitoring device placed on the hand of a user.

FIG. 9 is a schematic view of another alternative embodiment of the monitoring device utilized with a mobile communication device.

FIG. 10 is a flow chart of a method of monitoring.

FIG. 11 is an image of an activity log of information obtained from a monitoring device.

FIG. 12 is an image of calorie information obtained from a monitoring device.

FIG. 13 is an image of a time dependent optical waveform generated by an optical sensor with placement of the optical sensor on a lower portion of an index finger.

FIG. 14 is an image of a time dependent optical waveform generated by an optical sensor with placement of the optical sensor on a tip of an index finger.

FIG. 15 is an image of a time dependent optical waveform generated by an optical sensor with placement of the optical sensor on a nail of a thumb.

FIG. 16 is an image of a time dependent optical waveform generated by an optical sensor with placement of the optical sensor on a base of a pinky finger.

FIG. 17 is an image of a time dependent optical waveform generated by an optical sensor with placement of the optical sensor on a bottom of an index finger.

FIG. 18 is an image of a time dependent optical waveform generated by an optical sensor with placement of the optical sensor on a lower portion of an index finger.

FIG. 19 is a schematic diagram of combined circuit assembly and display member utilized with the monitoring device.

FIG. 20 is an isolated side view of a control component utilized with a monitoring device.

FIG. 21 is an isolated top plan view of the control component of FIG. 20.

FIG. 22 is a flow chart for using the control component to input information and output information on a display of the monitoring device.

### DETAILED DESCRIPTION OF THE INVENTION

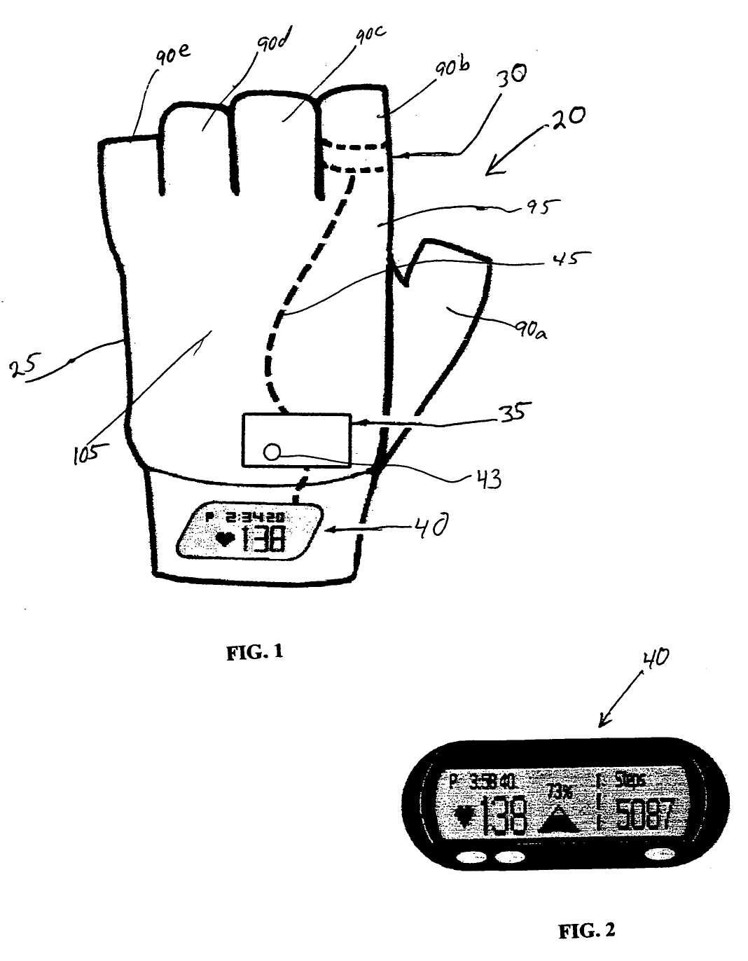

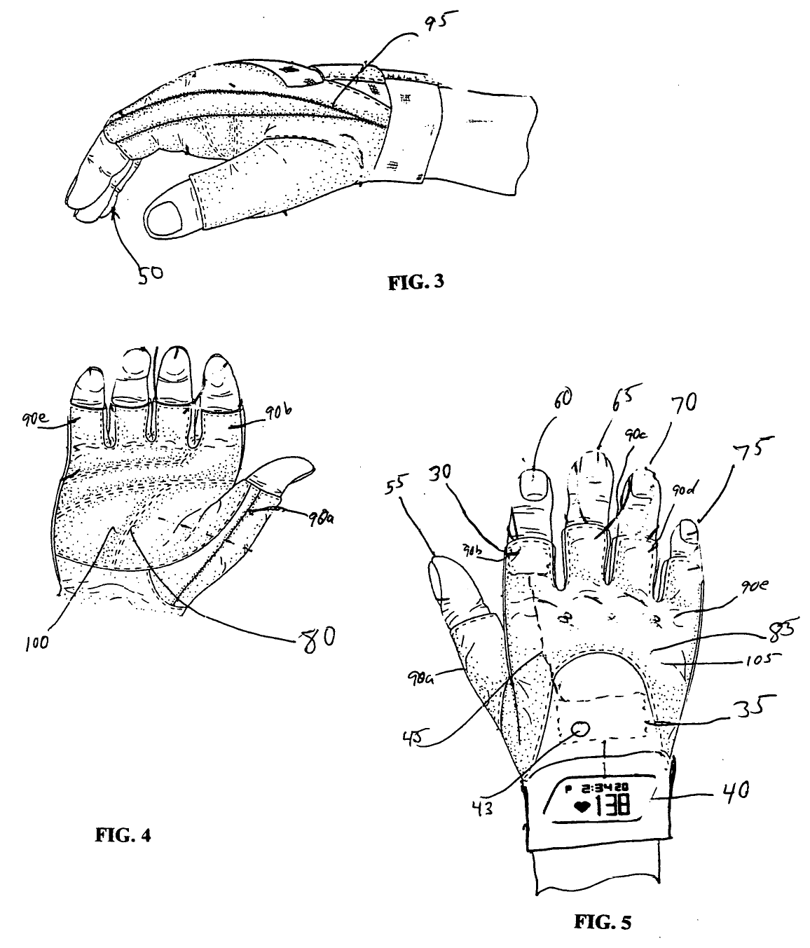

As shown in FIGS. 1 and 3-5, a monitoring device is generally designated 20. The monitoring device 20 preferably includes a glove 25, an optical sensor 30, a circuitry assembly 35, a display member 40, a control component 43 and a connection wire 45. The monitoring device 20 is preferably placed on a user's hand 50. The user's thumb 50, index finger 60, middle finger 65, ring finger 70 and pinky finger 75 are preferably placed through corresponding sleeves 90a-e of a body 95 of the glove 25. The body 95 also preferably has a palm portion 100 that covers the user's palm 80 and a back portion 105 that covers the back 85 of the user's hand 50.

It is desirous to adapt the glove 25 to the anatomy of the hand 50. The glove 25 is preferably composed of leather, synthetic leather, LYCRA, another similar material, or a combination thereof. The palm portion 100 has an interior surface and an exterior surface. The back portion 105 has an interior surface and an exterior surface. The finger end includes the four finger sleeves 90b-e and the thumb sleeve 90a. The little finger side has an interior surface and an exterior surface. The wrist end has an interior surface and an exterior surface. The glove 25 preferably has a mass ranging from 5 grams to 50 grams. Preferably, the lower the mass of the glove, the more comfort to the user.

The interior surface of the palm portion 100, the interior surface of the back portion 105, an interior surface of the thumb sleeve 90a, the interior surface of the pinky finger sleeve 90e, and the interior surface of the wrist end are operably connected to one another to thereby define an overall interior area of the internal sleeve of the glove 25. A user's hand 50, which includes a wrist, a palm 80, knuckle area, and fingers 60, 65, 70 and 75, is placed within the overall interior area of the internal sleeve when in use.

The exterior surface of the palm portion 100, the exterior surface of the back portion 105, an exterior surface of the thumb sleeve 90a, the exterior surface of the pinky finger sleeve 90e, and the exterior surface of the wrist end are operably connected to one another to thereby define an overall exterior area of the glove 25. The overall exterior area of the glove 25 is the area that is apparent on the user's hand 50.

In a preferred embodiment, each one of the four finger sleeves 90b-e of the finger end have an open end and a length such that each finger 60, 65, 70 and 75 of the user's hand 50 is bare from a finger tip area to a point between the knuckle area and a first finger joint of the finger. However, those skilled in the pertinent art will recognize that the glove 25 may be constructed such that some or all of the four finger sleeves 90b-e have any length, from no length at all to full-finger length. Full length four finger sleeves 90b-e may be close-ended rather than open-ended.

In similar fashion, the thumb sleeve 90a typically has an open end and a length such that a thumb 55 of the user's hand 50 protrudes from the thumb sleeve 90a. However, those skilled in the pertinent art will recognize that the glove 25 may be constructed such that the thumb sleeve 90a has any length, from no length at all to full-thumb length. Full length thumb sleeve 90a may be close-ended rather than open-ended.

In a preferred embodiment, the optical sensor 30 is a photodetector 130 and a single light emitting diode (“LED”) 135 transmitting light at a wavelength of approximately 660 nanometers. As the heart pumps blood through the arteries in the user's ear, blood cells absorb and transmit varying amounts of the light depending on how much oxygen binds to the cells' hemoglobin. The photodetector 30, which is typically a photodiode, detects transmission at the red wavelengths, and in response generates a radiation-induced signal.

Alternatively, the optical sensor 30 is a pulse oximetry device with a light source 135 that typically includes LEDs that generate both red (λ˜660 nm) and infrared (λ˜900 nm) radiation. As the heart pumps blood through the arteries in the hand of the user, blood cells absorb and transmit varying amounts of the red and infrared radiation depending on how much oxygen binds to the cells' hemoglobin. The photodetector 130, which is typically a photodiode, detects transmission at the red and infrared wavelengths, and in response generates a radiation-induced signal.

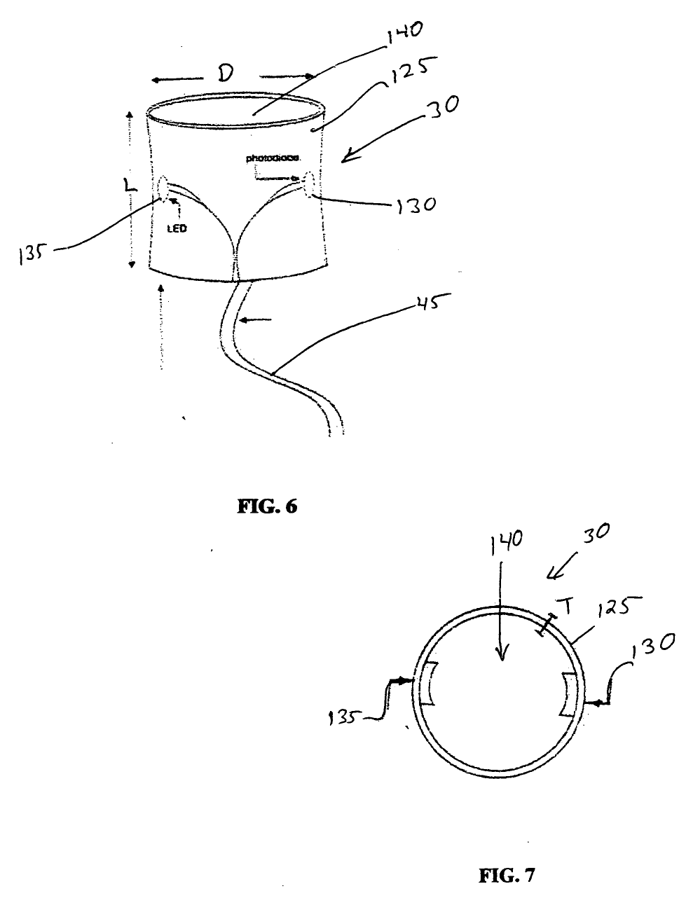

FIGS. 6 and 7 illustrate an optical sensor 30 that is utilized with the present invention. The optical sensor 30 has a body 125 with a photo-detector 130 and a light source 135 embedded therein. The body 125 is preferably composed of a neoprene material. An aperture 140 allows for placement of the device over a user's finger. The body 125 preferably has a thickness, “T”, that preferably ranges from 1.5 to 4.0 millimeters, and more preferably from 2.0 to 3.0 millimeters. The body 125 has a length, “L”, that preferably ranges from 1.5 to 3.0 centimeters, and more preferably from 2.0 to 2.5 centimeters. The aperture 140 of the body 125 has a diameter, “D”, that preferably ranges from 1.25 to 3.0 centimeters, and more preferably from 1.50 to 2.0 centimeters, and is most preferably 1.75 centimeters.

Alternatively, the optical sensor 30 is pulse oximetry device comprising the photo-detector 130, a first light source 125 and a second light source 125a, not shown, all of which are embedded directly in a sleeve 90 of the body 95 of the glove 25. In this embodiment, the first light source 125 emits light in an infrared range (λ˜900 nm) and the second light source 125a emits light in a red range (λ˜630 nm). In either embodiment, placement of the optical sensor 30 is preferably in a lower portion of the user's index finger 60.

The light source 135 typically is a light-emitting diode that emits light in a range from 600 nanometers to 110 nanometers. As the heart pumps blood through the patient's finger, blood cells absorb and transmit varying amounts of the red and infrared radiation depending on how much oxygen binds to the cells' hemoglobin. The photodetector 30, which is typically a photodiode, detects transmission at the red and infrared wavelengths, and in response generates a radiation-induced current that travels through the connection wire 45 to the circuitry assembly 35 embedded within the back portion 105 of the body 95 of the glove 25.

A preferred photodetector is a light-to-voltage photodetector such as the TSL260R and TSL261, TSL261R photodetectors available from TAOS, Inc of Plano Tex. Alternatively, the photodetector is a light-to-frequency photodetector such as the TSL245R, which is also available from TAOS, Inc. The light-to-voltage photodetectors have an integrated transimpedance amplifier on a single monolithic integrated circuit, which reduces the need for ambient light filtering. The TSL261 photodetector preferably operates at a wavelength greater than 750 nanometers, and optimally at 940 nanometers, which would preferably have a LED that radiates light at those wavelengths.

In a preferred embodiment, the circuit assembly 35 is flexible to allow for the contour of the user's hand and movement thereof. Preferably the dimensions of a board of the circuit assembly 35 are approximately 39 millimeters (length) by approximately 21 millimeters (width) by 0.5 millimeters (thickness).

Alternatively, the circuitry assembly 35 includes a flexible microprocessor board and a flexible pulse oximetry board. A preferred pulse oximetry board is a BCI MICRO POWER oximetry board, which is a low power, micro-size easily integrated board which provides blood oxygenation level, pulse rate (heart rate), signal strength bargraph, plethysmogram and status bits data. The size of the board is preferably 25.4 millimeters (length)×12.7 millimeters (width)×5 millimeters (thickness). The microprocessor board receives data from the pulse oximetry board and processes the data to display on the display member 40. The microprocessor can also store data. The microprocessor can process the data to display pulse rate, blood oxygenation levels, calories expended by the user of a pre-set time period, target zone activity, time and dynamic blood pressure. Alternatively, the circuitry assembly 35 is a single board with a pulse oximetry circuit and a microprocessor.

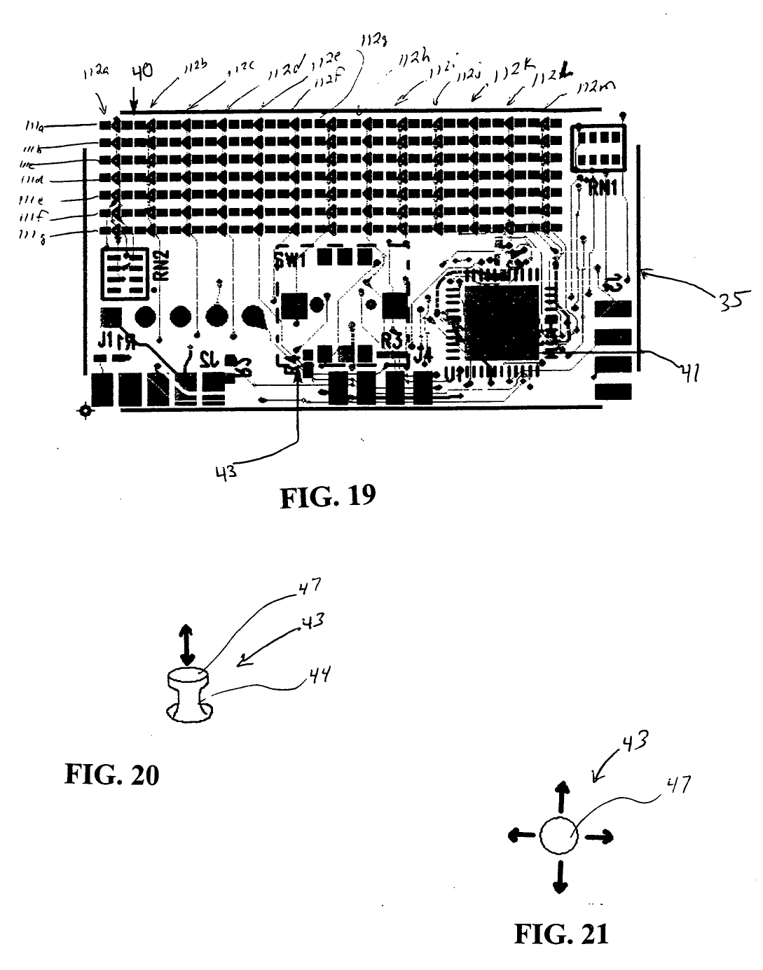

The display member 40 is preferably a liquid crystal display (“LCD”). Alternatively, the display member 40 is a light emitting diode (“LED”) or other similar display device. As shown in FIG. 19, the display member 40 is an LED array which preferably has seven rows 111a-111g and thirteen columns 112a-112m. The LED array allows for each column to be illuminated separately thereby giving the appearance of a moving display. For example, if the term “200 calories expended” is displayed on the display member 40, the “2” of the “200” would preferably first appear in column 112m and then subsequently in each of the other columns 112l-112a, from the right-most column to the left-most column thereby giving the appearance of the term scrolling along the display member 40. The terms or words alternatively scroll from left to right. Still alternatively, all of the columns are illuminated at once or all flash in strobe like manner. Those skilled in the pertinent art will recognize alternative methods of displaying information on the display member 40 without departing from the scope and spirit of the present invention.

As shown in FIG. 19, the display member 40 is combined with the circuit assembly 35. A microcontroller 41 processes the signal generated from the optical sensor 30 to generate the plurality of vital sign information for the user which is displayed on the display member 40. The control component 43 is connected to the circuit assembly 35 to control the input of information and the output of information displayed on the display member 40.

FIGS. 20-21 illustrate an isolated view of a preferred embodiment of the control component 43. The control component 43 preferably has a body 44 with a top 47. The body 44 preferably has a shape which minimizes mass and is easily operated by the user. The control component 43 is preferably a button or “joystick” that is capable of multiple dimensional movement such as being compressible up and down as indicated by the arrow in FIG. 20 or in an X-Y movement as indicated by the arrows in FIG. 21. The multiple dimensional movement of the control component 43 allows for the user to enter or select functions and scroll through menus which are displayed on the display member 40, as discussed below.

The monitoring device 20 is preferably powered by a battery 89, not shown, connected to the circuit assembly 35. The battery 89 is preferably a lithium ion rechargeable battery such as available from NEC-Tokin. The circuit assembly 35 preferably requires 5 volts and draws a current of 20-to 40 milliamps. The battery 89 preferably provides at least 900 milliamp hours of power to the monitoring device 20.

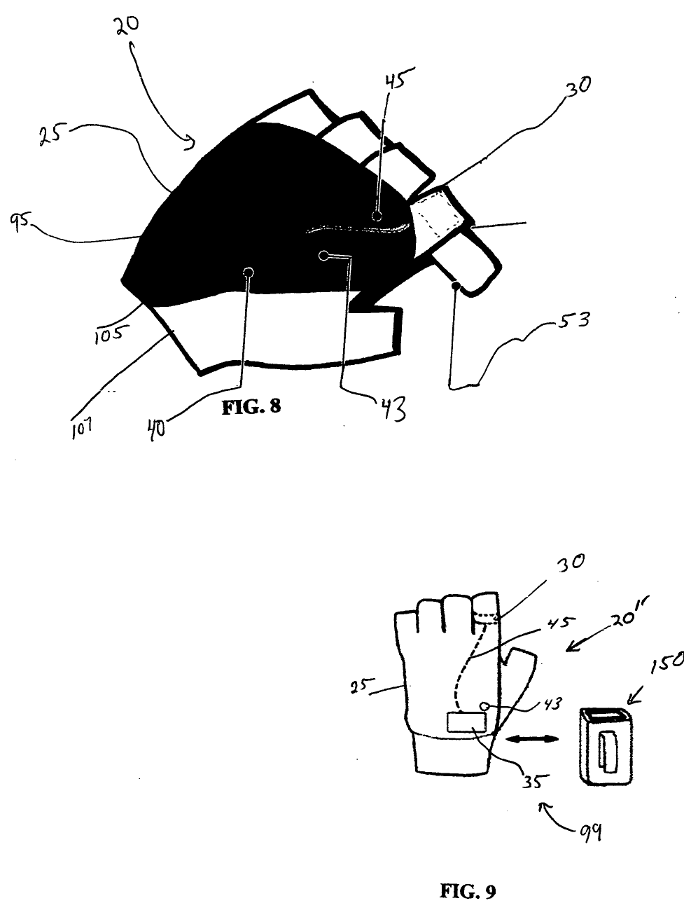

FIG. 8 illustrates an alternative embodiment of the monitoring device 20. In this embodiment the display member 40 is preferably angled at an angle ranging form 20 to 70 degrees relative to an edge 107 of the glove 25, more preferably ranging from 30 to 60 degrees relative to the edge 107, and most preferably 45 degrees relative to the edge 107.

Yet another embodiment is illustrated in FIG. 9. In this embodiment, the monitoring device 20″ is similar to the monitoring device 20 of FIG. 1. However, a short range wireless transceiver is included in the circuitry assembly 35 for transmitting information processed from the pulse oximetry device 30 to a handheld device 150 or a computer, not shown, to form a system 99. The display member 40 is optional in this embodiment.

The short-range wireless transceiver is preferably a transmitter operating on a wireless protocol, e.g. Bluetooth™, part-15, or 802.11. “Part-15” refers to a conventional low-power, short-range wireless protocol, such as that used in cordless telephones. The short-range wireless transmitter (e.g., a Bluetooth™ transmitter) receives information from the microprocessor and transmits this information in the form of a packet through an antenna. The external laptop computer or hand-held device 150 features a similar antenna coupled to a matched wireless, short-range receiver that receives the packet. In certain embodiments, the hand-held device 150 is a cellular telephone with a Bluetooth circuit integrated directly into a chipset used in the cellular telephone. In this case, the cellular telephone may include a software application that receives, processes, and displays the information. The secondary wireless component may also include a long-range wireless transmitter that transmits information over a terrestrial, satellite, or 802.11-based wireless network. Suitable networks include those operating at least one of the following protocols: CDMA, GSM, GPRS, Mobitex, DataTac, iDEN, and analogs and derivatives thereof. Alternatively, the handheld device 150 is a pager or PDA.

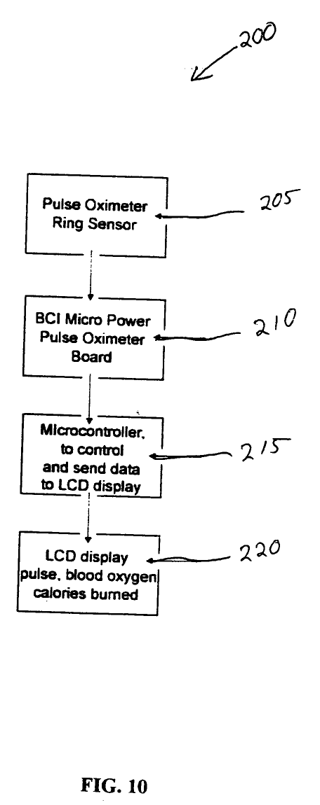

As shown in FIG. 10, a general method is indicated as 200. At block 200, the light source 135 transmits red and infrared light through a finger of the user. The photo-detector 130 detects the light. The pulse rate is determined by the signals received by the photo-detector 130. The ratio of the fluctuation of the red and infrared light signals is used to calculate the blood oxygen saturation level of the user. An optical sensor 30 with a photodetector 130 and single LED 135 is preferably utilized. Alternatively, a pulse oximetry device with two LEDs and a photodetector is utilized.

At block 210, this information is sent to pulse oximetry board in the circuitry assembly 35 for creation of blood oxygenation level, pulse rate, signal strength bargraph, plethysmogram and status bits data. At block 215, the microprocessor further processes the information to display pulse rate, blood oxygenation levels, calories expended by the user of a pre-set time period, target zones of activity, time and dynamic blood pressure. At block 220, the information is displayed on the display member.

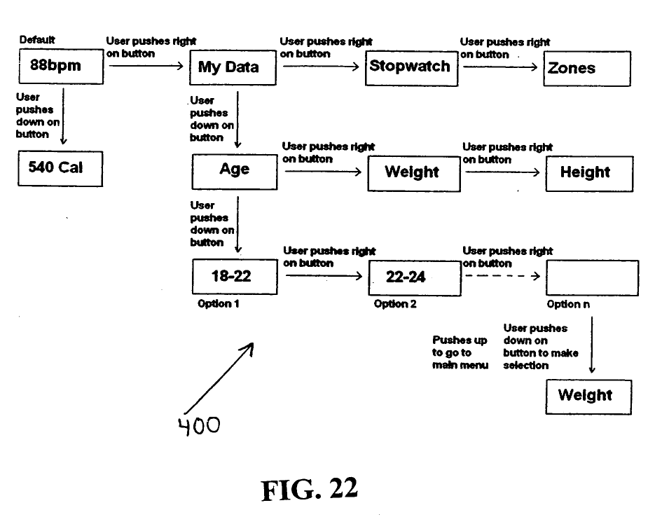

A flow chart diagram 400 for using the control component 43 with the display member 40 is shown in FIG. 22. As mentioned above, the control component 43 allows a user to scroll and select from terms displayed on the display member 40. User inputs preferably include age, gender, weight, height and resting heart rate which can be inputted and stored in a memory of the circuit assembly 35. The real time heart rate of the user is preferably displayed as a default display, and the user's real time heart rate is preferably updated every ten seconds based on measurements from the optical sensor 30. Based on the user inputs, the calories expended by the user for a set time period are calculated and displayed on the display member 40 as desired by the user using the control component 43. The monitoring device 20 will also preferably include a conventional stop watch function, which is displayed on the display member 40 as desired by the user. The display member 40 preferably displays a visual alert when a user enters or exits a target zone such as a cardio zone or fat burning zone. The monitoring device 20 optionally includes an audio alert for entering or exiting such target zones.

The user can toggle the control component 43 to maneuver between the user's real-time heart rate and real time calories expended by the user during a set time period. The user can also scroll through a menu-like display on the display member 40 and enter options by pushing downward on the control component 43. The options can preferably include a “My Data” section which the user inputs by scrolling and selection an option by pushing downward, such as selecting between male and female for gender. The user can also select target zones by scrolling through a different section of the menu. As discussed below, each target zone is calculated using a formula based upon the user's personal data. In operation, when a specific target zone is selected, a visual alert in the form of a specific display such as an icon-like picture is displayed on the display member 40 to demonstrate that the user is now in the specified target zone. The icon preferably blinks for a set period of time such as ten seconds. Those skilled in the pertinent art will recognize that other options may be included on the menu-like display without departing from the spirit and scope of the present invention.

In yet an alternative embodiment, an accelerometer, not shown, is embedded within the body 95 of the glove 25 and connected to the circuitry assembly 35 in order to provide information on the distance traveled by the user. In a preferred embodiment, the accelerometer is a multiple-axis accelerometer, such as the ADXL202 made by Analog Devices of Norwood, Mass. This device is a standard micro-electronic-machine (“MEMs”) module that measures acceleration and deceleration using an array of silicon-based structures.

In yet another embodiment, the monitoring device 20 comprises a first thermistor, not shown, for measuring the temperature of the user's skin and a second thermistor, not shown, for measuring the temperate of the air. The temperature readings are displayed on the display member 40 and the skin temperature is preferably utilized in further determining the calories expended by the user during a set time period. One such commercially available thermistor is sold under the brand LM34 from National Semiconductor of Santa Clara, Calif. A microcontroller that is utilized with the thermistor is sold under the brand name ATMega 8535 by Atmel of San Jose, Calif.

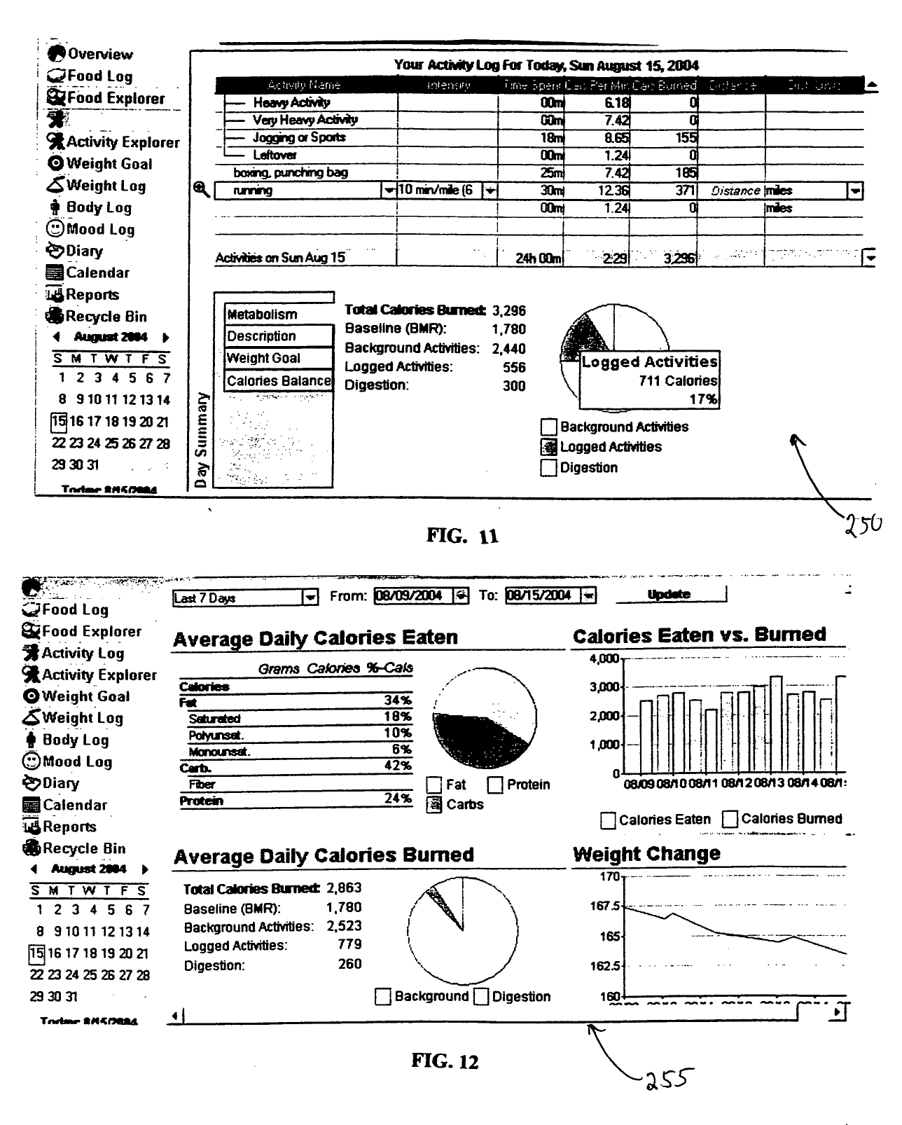

The monitoring device 20 may also be able to download the information to a computer for further processing and storage of information. The download may be wireless or through cable connection. The information can generate an activity log 250 such as shown in FIG. 11, or a calorie chart 255 such as shown in FIG. 12.

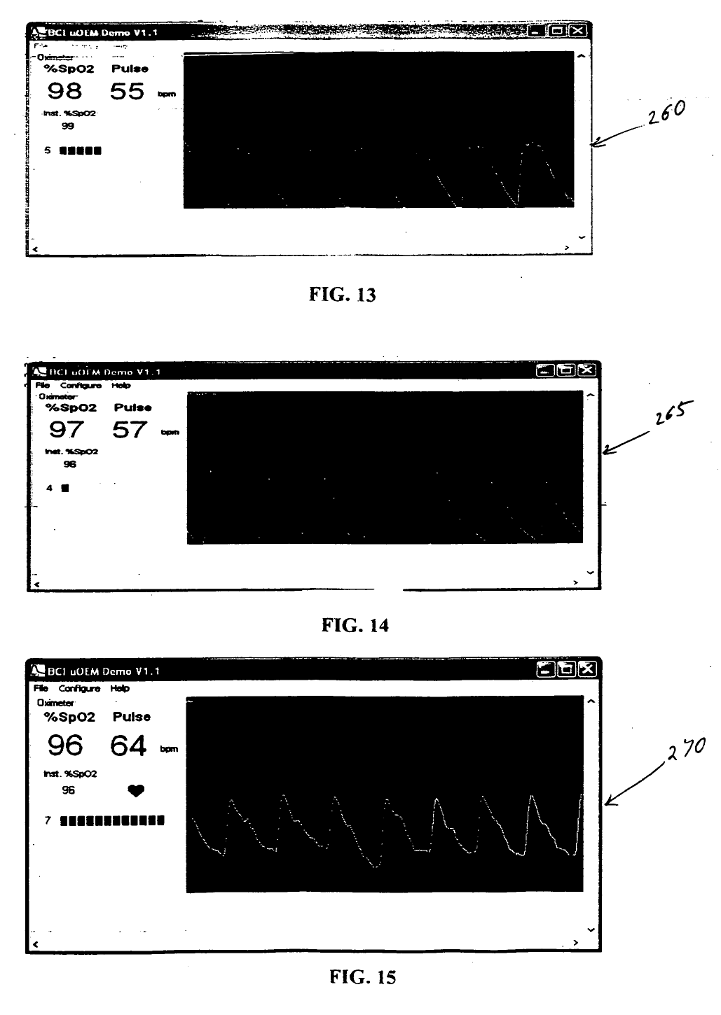

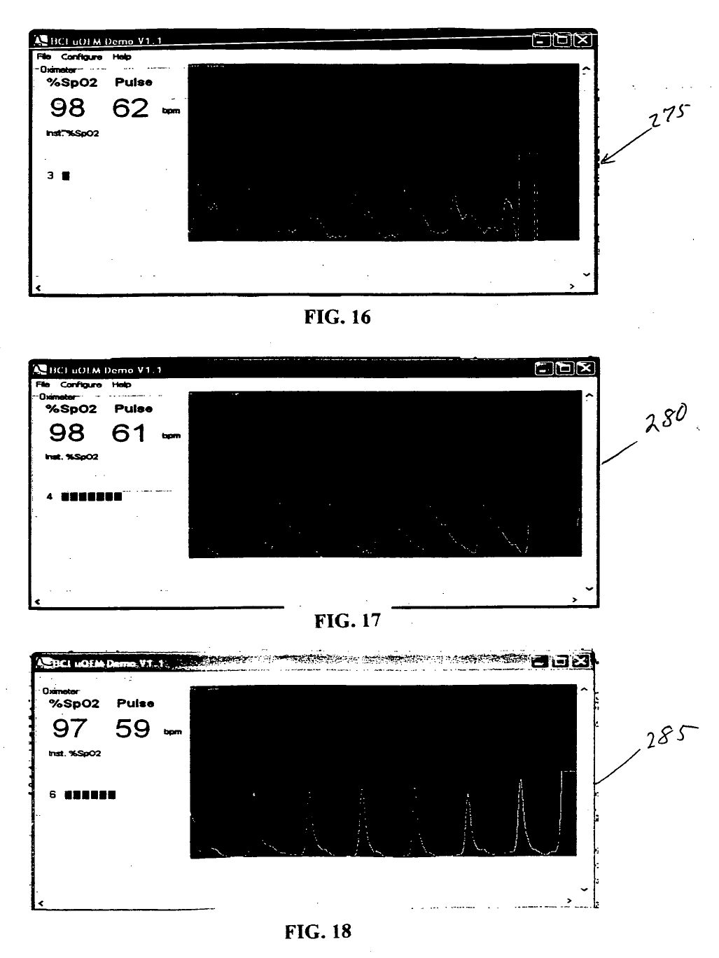

FIGS. 13-18 illustrate images 260-285 of optical waveforms generated by the pulse oximetry device 30 positioned on various fingers and at various positions of the fingers. The positioning of the light source 135 and detector 130 at the tip of the index finger provide a sharp and strong waveform as illustrated in FIG. 14. Placement of the device 30 at the base of the pink finger provided a weak and undefined waveform, as shown in FIG. 16.

The microprocessor can use various methods to calculate calories burned by a user. One such method uses the Harris-Benedict formula. Other methods are set forth at www.unu.edu/unupress/food2/ which relevant parts are hereby incorporated by reference. The Harris-Benedict formula uses the factors of height, weight, age, and sex to determine basal metabolic rate (BMR). This equation is very accurate in all but the extremely muscular (will underestimate calorie needs) and the extremely overweight (will overestimate caloric needs) user.

The equations for men and women are set forth below:
Men: BMR=66+(13.7×mass (kg))+(5×height (cm))−(6.8×age (years))
Women: BMR=655+(9.6×mass)+(1.8×height)−(4.7×age)

The calories burned are calculated by multiplying the BMR by the following appropriate activity factor: sedentary; lightly active; moderately active; very active; and extra active.
Sedentary=BMR multiplied by 1.2 (little or no exercise, desk job)
Lightly active=BMR multiplied by 1.375 (light exercise/sports 1-3 days/wk)
Moderately Active=BMR multiplied by 1.55 (moderate exercise/sports 3-5 days/wk)
Very active=BMR multiplied by 1.725 (hard exercise/sports 6-7 days/wk)
Extra Active=BMR multiplied by 1.9 (hard daily exercise/sports & physical job or 2× day training, marathon, football camp, contest, etc.)

Various target zones may also be calculated by the microprocessor. These target zones include: fat burn zone; cardio zone; moderate activity zone; weight management zone; aerobic zone; anaerobic threshold zone; and red-line zone.

Fat Burn Zone=(220−age)×60% & 70%

An example for a thirty-eight year old female:

(220−38)×0.6=109
(220−38)×0.7=127
Fat Burn Zone between 109 to 127 heart beats per minute.

Cardio Zone=(220−your age)×70% & 80%

An example for a thirty-eight year old female:

(220−38)×0.7=127
(220−38)×0.8=146
Cardio zone is between 127 & 146 heart beats per minute.

Moderate Activity Zone, at 50 to 60 percent of your maximum heart rate, burns fat more readily than carbohydrates. That is the zone one should exercise at if one wants slow, even conditioning with little pain or strain.

Weight Management Zone, at 60 to 70 percent of maximum, strengthens ones heart and burns sufficient calories to lower one's body weight.

Aerobic Zone, at 70 to 80 percent of maximum, not only strengthens one's heart but also trains one's body to process oxygen more efficiently, improving endurance.

Anaerobic Threshold Zone, at 80 to 90 percent of maximum, improves one's ability to rid one's body of the lactic-acid buildup that leads to muscles ache near one's performance limit. Over time, training in this zone will raise one's limit.

Red-Line Zone, at 90 to 100 percent of maximum, is where serious athletes train when they are striving for speed instead of endurance.

### EXAMPLE ONE

Female, 30 yrs old, height 167.6 centimeters, weight 54.5 kilograms.
The BMR=655+523+302−141=1339 calories/day.
The BMR is 1339 calories per day. The activity level is moderately active (work out 3-4 times per week). The activity factor is 1.55. The TDEE=1.55×1339=2075 calories/day. TDEE is calculated by multiplying the BMR of the user by the activity multiplier of the user.

A system 500 may use the heart rate to dynamically determine an activity level and periodically recalculate the calories burned based upon that factor. An example of such an activity level look up table might be as follows:
Activity/Intensity Multiplier Based on Heart Rate
Sedentary=BMR×1.2 (little or no exercise, average heart rate 65-75 bpm or lower)
Lightly active=BMR×3.5 (light exercise, 75 bpm-115 bpm)
Mod. active=BMR×5.75 (moderate exercise, 115-140 pm)
Very active=BMR×9.25 (hard exercise, 140-175 bpm)
Extra active=BMR×13 (175 bpm−maximum heart rate as calculated with MHR formula)

For example, while sitting at a desk, a man in the above example might have a heart rate of between 65 and 75 beats per minute (BPM). (The average heart rate for an adult is between 65 and 75 beats per minute.) Based on this dynamically updated heart rate his activity level might be considered sedentary. If the heart rate remained in this range for 30 minutes, based on the Harris-Benedict formula he would have expended 1.34 calories a minute×1.2 (activity level)×30 minutes, which is equal to 48.24 calories burned.

If the man were to run a mile for 30 minutes, with a heart rate ranging between 120 and 130 bpm, his activity level might be considered very active. His caloric expenditure would be 1.34 calories a minute×9.25 (activity level)×30 minutes, which is equal to 371.85.

Another equation is weight multiplied by time multiplied by an activity factor multiplied by 0.000119.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention:

1. A method of monitoring a user's vital signs, the method comprising:
generating a signal corresponding to the flow of blood through an artery of the user, the signal generated from an optical sensor connected to a body of a glove;
generating heart rate data of the user and an oxygen saturation level data of the user from the signal generated by the optical sensor;
processing the heart rate data and oxygen saturation level data of the user at a microcontroller disposed with the body of the glove for analysis of calories expended by the user and for display of a plurality of the user's vital signs; and
displaying the calories expended by the user, the user's heart rate or the user's blood oxygen saturation level on a display member disposed on an exterior surface of the body of the glove which is controlled by the user using a control component extending from the body of the glove.

2. The method according to claim 1 further comprising measuring the distance traveled by the user during a time period using an accelerometer disposed on the glove body, and displaying the distance traveled by the user on the display member.

3. The method according to claim 1 further comprising determining the user's dynamic blood pressure from the heart rate date and the oxygen saturation level of the user, and displaying the user's dynamic blood pressure on the display member.

4. The method according to claim 1 further comprising wirelessly transmitting the calories expended by the user, the user's heart rate and the user's blood oxygen saturation level from a wireless transceiver embedded within the body of the glove to a mobile communication device or a computer.

5. A monitoring device for monitoring the health of an user, the monitoring device comprising:
a glove;
means for measuring blood flow through an artery of a finger of the user, the measuring means connected to the glove;
means for calculating calories expended by the user during a time period, the calculating means disposed on the glove;
means for visually displaying the calories expended by the user, the visually displaying means attached to an exterior surface of the glove; and
means for controlling the input information and the output of information displayed on the visually displaying means, the controlling means extending from the exterior surface of the glove.

6. The monitoring device according to claim 5 further comprising means for determining the pulse rate of the user.

7. The monitoring device according to claim 5 wherein the glove comprises a body, a plurality of finger sleeves, a thumb sleeve, a palm portion and a back portion.

8. The monitoring device according to claim 5 wherein the controlling means is a joystick extending outward from the body of the glove, the joystick capable of multiple dimensional movement to input information and control the out of information on the visually displaying means.

9. The monitoring device according to claim 5 further comprising means for wireless transmitting a plurality of the user's vital signs to a mobile communication device or computer.

10. The monitoring device according to claim 5 wherein the measuring means is an optical sensor comprising a light-to-voltage photodetector capable of transmitting a digital signal, and at least one light emitting diode capable of radiating light ranging from 600 nanometers to 1100 nanometers.

11. The monitoring device according to claim 5 wherein the measuring means is a pulse oximetry sensor comprising a light-to-voltage photodetector capable of transmitting a digital signal, first light emitting diode capable of radiating red light and a second light emitting diode capable of emitting infrared light.

12. A monitoring device for monitoring the health of a user, the monitoring device comprising:
a glove;
an optical sensor connected to the glove;
a circuitry assembly embedded within the glove;
a display member attached to an exterior surface of the glove;
a control component extending from the exterior surface of the glove, the control component controlling the input of information and the output of information displayed on the display member.

13. The monitoring device according to claim 12 wherein the optical sensor comprises a light-to-voltage photodetector capable of transmitting a digital signal, and at least one light emitting diode capable of radiating light ranging from 600 nanometers to 1100 nanometers.

14. The monitoring device according to claim 12 wherein the optical sensor is a pulse oximetry sensor comprising a light-to-voltage photodetector capable of transmitting a digital signal, first light emitting diode capable of radiating red light and a second light emitting diode capable of emitting infrared light.

15. The monitoring device according to claim 12 wherein the glove comprises a body, a plurality of finger sleeves, a thumb sleeve, a palm portion and a back portion.

16. The monitoring device according to claim 15 wherein the optical sensor is embedded within one of the plurality of finger sleeves of the glove and the display member is attached to an exterior surface of the back portion of the glove.

17. The monitoring device according to claim 14 wherein the circuitry assembly comprises a pulse oximetry board and a microprocessor.

18. The monitoring device according to claim 12 wherein a plurality of the user's vital signs are displayed on the display member, the plurality of the user's vital signs comprises calories expended by the user, the user's heart rate, the user's blood oxygen saturation level, a target zone, distance traveled and dynamic blood pressure.

19. The monitoring device to claim 12 wherein the circuit assembly further comprises an accelerometer for measuring the distance traveled by the users, the display member capable of displaying the distance traveled by the user.

20. The monitoring device according to claim 12 wherein the optical sensor comprises a light-to-frequency photodetector capable of transmitting a digital signal, and at least one light emitting diode capable of radiating light ranging from 600 nanometers to 1100 nanometers.
